(19) United States
(12) Patent Application Publication
(10) Pub. No.: US 2025/0239364 A1
(43) Pub. Date: Jul. 24, 2025

(54) DIAGNOSTIC SYSTEM AND METHOD

(71) Applicant: NUMARES AG, Regensburg (DE)

(72) Inventors: Andreas WOLF, Regensburg (DE); Andreas FALTERMEIER, Perkam (DE); Volker PFAHLERT, Kandern (DE)

(73) Assignee: NUMARES AG, Regensburg (DE)

(21) Appl. No.: 18/703,380

(22) PCT Filed: Oct. 11, 2022
(86) PCT No.: PCT/EP2022/078270

§ 371 Date: Apr. 22, 2024

(30) Foreign Application Priority Data
Oct. 21, 2021 (EP) 21204066.1

(51) Int. Cl. G16H 50/20; G16H 10/40; G16H 10/60; G16H 30/40

(52) U.S. Cl. CPC G16H 50/20; G16H 10/40; G16H 10/60; G16H 30/40

(57) ABSTRACT

A method for analysis in medical diagnostics in accordance with the present invention can comprise the steps of collecting measurement data, analysing analysis data corresponding to at least a portion of the measurement data and thereby generating an analysis result, and/or storing the measurement data and/or analysis data and/or analysis result in a data storage system. The step of storing the measurement data and/or analysis data and/or analysis result in the data storage system can comprise transmitting a request for storing the measurement data and/or analysis data and/or analysis result to an interface module, and/or in response, the interface module prompting the measurement and/or analysis data and/or analysis result data to be stored in the data storage system. The invention is directed to a system for analysis in medical diagnostics, wherein the system can comprise a main module, at least one measurement device configured to provide measurement data on a sample and/or subject, at least one analysis module configured to analyse analysis data and provide an analysis result, a data storage system configured to store and provide data, and/or an interface module. The system can additionally or alternatively comprise a plurality of software-based toolsets in a data storage system wherein the system is configured to transfer one or more toolset(s) to a user in response to a query transfer. The data storage system can be configured to store the measurement data and/or analysis data and/or analysis result. The system and method can be configured for spectral analysis in medical diagnostics, wherein the medical diagnostics can be metabolite diagnostics.

### FIELD

The present invention generally relates to systems and methods for diagnostics. In particular, the present invention relates to medical and clinical diagnostics, for example for diagnosis, screening and/or monitoring of patients, as well as research purposes.

### INTRODUCTION

Known diagnostic systems typically provide highly specific testing capabilities based on specific measurements. For example, a diagnostic system is provided by means of a modular software on a stand-alone processing device, e.g. a computer, in combination with a respective measurement device. Such a diagnostic system is configured to acquire measurement data, e.g. on a patient's blood or urine sample, with a designated measurement device, e.g. a nuclear magnetic resonance (NMR) spectrometer, a mass spectrometer or other suitable devices. The measurement data is received and processed by the processing device and stored in a local database on said processing device. Tests are typically software modules that are applied to the gathered measurement data, such that a single set of measurement data corresponding to a single sample may for example be analysed with multiple, different tests. Each test typically provides a designated result, e.g. a score, that may for example be indicative of a pre-defined disease. Generally, the software-based tests analyse the measurement data to become diagnostics data and for example evaluate the presence or abundance of certain metabolites and/or metabolite constellations.

While such systems may provide basic diagnostic functionalities, they may also comprise certain disadvantages: Known diagnostics systems may be particularly designed for local operation. That is, they may comprise designated standalone desktop applications typically designed for a specific type of measurements and installed on a designated Hardware system running a selected operating system. In other words, such systems may be highly specialised and only designed for local use. Thus, known diagnostic systems may disadvantageously lack connectivity and flexibility of operation and accessibility of measurement data and results. For example, measurement data of different devices may not readily be accessible to a single test as results of different devices are locally stored in different, and thus independent, databases. Typically, each database may only be locally accessible via the corresponding software module. Even if a test would have access to data of multiple databases, the internal structure and particularly the referencing within each of the independent databases may be incompatible or at least require significant additional effort to ensure correct association and matching of different datasets.

Furthermore, each software-based test is typically provided by a separate software module, e.g. a plug in, comprising all algorithms necessary to process and analyse the measurement data. This may disadvantageously lead to redundancies between different tests installed on the same device. Furthermore, it may not easily be possible to allow a user to access at least some of the algorithms used to process and analyse the data, which may, however, generally be desirable for a user to develop new tests.

### SUMMARY

In light of the above, it is an object to overcome or at least alleviate the shortcomings and disadvantages of the prior art. That is, it is an object of the present invention to provide an improved system for analysis in medical diagnostics and a corresponding method.

These objects are met by the present invention.

In the following, a system and a method are exemplified according to the present invention. The method also comprises the system features in terms of method steps and vice-versa, where not explicitly mentioned.

In more detail, the invention is directed to a system for analysis in medical diagnostics, wherein the system can comprise a main module, at least one measurement device configured to provide measurement data on a sample and/or subject, at least one analysis module configured to analyse analysis data and provide an analysis result, a data storage system configured to store and provide data, and/or an interface module.

The term module is intended to mean an integral entity, preferably at one location and/or a multi-component entity that comprises two or more components that can even be spread among different locations. Just one out or a number of examples are cloud-computing components that can be spread around and can be part of a module.

The system can additionally or alternatively comprise a plurality of software-based toolsets in a data storage system wherein the system is configured to transfer one or more toolset(s) to a user in response to a query transfer.

The data storage system can be configured to store the measurement data and/or analysis data and/or analysis result.

The system can be configured for spectral analysis in medical diagnostics, wherein the medical diagnostics can be metabolite diagnostics.

The medical diagnostics can comprise diagnostics of human samples, animal samples and/or plant samples.

The at least one measurement device can comprise an imaging device, wherein the imaging device can be at least one of a computer tomography scanner and a magnetic resonance imaging scanner.

The at least one measurement device can comprise a spectrometer, a mass spectrometer and/or a nuclear magnetic resonance (NMR) spectrometer.

The measurement data can comprise NMR data that can comprise at least one NMR spectrum.

The at least one measurement device can be configured to communicate with the interface module.

The at least one measurement device can be configured to exchange data with the interface module or more specifically to send data to and receive data from the interface module. In particular, a device, module and/or component that is said to be configured to communicate with the interface module is configured to transmit requests to and receive responses from the interface module.

The at least one measurement device can be configured to transmit measurement data to the interface module.

The at least one measurement device can be configured to exchange data with the interface module or more specifically to send data to and receive data from the interface module. In particular, a device, module and/or component that is said to be configured to communicate with the interface module is configured to transmit requests to and receive responses from the interface module.

The at least one measurement device can be configured to transmit measurement data to the interface module.

The at least one measurement device can be configured to directly and/or indirectly communicate with the main module and/or can be configured to transmit measurement data to the main module.

The main module can be configured to communicate with the interface module.

The main module can be configured to directly communicate with the at least one measurement device and/or to directly communicate with the at least one analysis module.

The at least one analysis module can be configured to communicate with the interface module and/or to directly communicate with the main module.

The interface module can be configured to transmit and receive data and/or to communicate with the at least one measurement device and/or with the main module and/or with the at least one analysis module and/or with the data storage system and/or with external devices and modules.

The interface module can comprise at least one communication endpoint.

The at least one of the at least one communication endpoint can be configured for providing access to the data storage system.

The system can also comprise a plurality of interface modules.

Each of the plurality of interface modules can comprise different communication endpoints. It can also comprise at least one application programming interface (API).

The data storage system can comprise at least one database and/or at least one data warehouse and/or at least one remote server. It can be or comprise a remote, web-based data storage system.

The analysis data comprises measurement data and/or subject data comprising information regarding the subject to which the sample is attributed.

A measurement can be performed by the measurement device to provide measurement data at least partially based on at least one measurement parameter.

The analysis data can comprise at least a subset of the at least one measurement parameter.

The measurement data can comprise information indicative for the presence and/or abundance of metabolites in the measured sample.

The analysis module can be configured to assess the presence or abundance of certain metabolites and/or metabolite constellations in the sample.

The analysis result can comprise a score indicative of a pre-defined condition that can alternatively or additionally be based on the assessment of the presence or abundance of certain metabolites and/or metabolite constellations in the sample.

The main module can be configured to trigger and/or initiate an analysis performed by at least one of the at least one analysis module.

The main module can also or alternatively be configured to provide analysis parameters to the analysis module directly and/or via the interface module and/or to provide the analysis data to the analysis module directly and/or via the interface module.

The main module can comprise a driver of at least one of the at least one measurement device and/or can be configured to control the at least one measurement device and/or to control the at least one measurement device by providing control data to the measurement device directly and/or via the interface module.

The main module can be configured to trigger and/or initiate a measurement of at least one of the at least one measurement device.

The main module can be configured to provide and/or control at least a part of the at least one measurement parameter for a measurement of at least one of the at least one measurement device. The system can comprise at least one processing unit.

The main module can be configured to run on at least one of the at least one processing unit.

Each of the at least one analysis module can be configured to run on at least one of the at least one processing unit, respectively.

The interface module can be configured to run on at least one of the at least one processing unit. The measurement device can comprise at least one of the at least one processing unit.

The data storage system can comprise at least one of the at least one processing unit.

The system can comprise a toolset, wherein the toolset can comprise building blocks for processing and/or analysing the measurement and/or analysis data. It can be comprised by the data storage.

The toolset can be or can comprise a software development kit (SDK).

The building blocks can comprise algorithms for processing and/or analysing the measurement and/or analysis data and/or pre-compiled routines and/or reusable components of programming code.

The toolset can comprise a documentation comprising information regarding the building blocks and/or an application programming interface. The toolset can further comprise the interface module.

The sample can be or can comprise a clinical sample, at least one of blood, salvia, excreta, body tissue and/or tissue fluids.

The main module can be configured to receive the user input from the user interface.

The system can further comprise a user interface that can be configured to receive user input that can further be configured to provide data to a user. The user interface can additionally or alternatively comprise a visual interface.

The user interface can be configured to provide visual data via the visual interface.

The main module can be configured to pre-process the measurement data.

The interface module can be configured for secure communication and/or to require authentication for any communication with the interface module. Additionally or alternatively, it can be configured to require an authentication token to be provided to the interface module when communicating with the interface module. Additionally or alternatively, it can be configured to require a digital signature to be provided to the interface module when communicating with the interface module.

Any or a part of the communication with the interface module can be encrypted.

The system can be configured to perform a method for analysis in medical diagnostics according to any part of the subsequent or before description and embodiments.

The system can also comprise an interface for connecting to a laboratory information management system (LIMS). It can also comprise a storage configured for a storing of a toolset by a user for downloading the toolset for a defined kind of measurement data and/or measurement. A toolset can thus be assigned to certain kind or results of measurement data and/or findings to be achieved.

A third party's platform for storing their toolset and/or measurement data can be provided as well. Any first or also third party can develop and/or apply and/or modify a toolset present and can store and potentially offer this toolset to others. There can be provided a third-party toolset database storing a modified toolset by a first user for downloading the modified first user toolset configured for a defined kind of measurement data.

The modified first user toolset can comprise at least one of a first user originated toolset developed by the first user and/or third party; a combination of toolsets and/or a modification of a toolset; and/or any one of measurement data, analysis data and analysis results established by the first user and/or the third party.

The modified first user toolset stored in the third-party toolset database can be made available for other third-party users. A plurality of modified first user toolsets can be catalogized in the third-party toolset database.

The system can also comprise an invoicing step on behalf of the first user to the third-party users when the third-party user downloads the first user toolset. The invoicing step on behalf of the first user can be internally split up the invoice to the benefit of the first user and an organizing entity. The organizing entity can thus receive a part of the invoice for providing and managing the infrastructure.

A method for analysis in medical diagnostics in accordance with the present invention can comprise the steps of collecting measurement data, analysing analysis data corresponding to at least a portion of the measurement data and thereby generating an analysis result, and/or storing the measurement data and/or analysis data and/or analysis result in a data storage system. The step of storing the measurement data and/or analysis data and/or analysis result in the data storage system can comprise transmitting a request for storing the measurement data and/or analysis data and/or analysis result to an interface module, and/or in response, the interface module prompting the measurement and/or analysis data and/or analysis result data to be stored in the data storage system.

A method for analysis in medical diagnostics can additionally or alternatively comprise the steps of providing a plurality of software-based toolsets in a data storage system, and in response to a query transfer one or more toolsets to a user.

The method can comprise spectral analysis in medical diagnostics, such as metabolite diagnostics. The medical diagnostics can comprise diagnostics of human samples, animal samples and/or plant samples.

The method can further comprise providing the sample, pre-processing the sample. The sample can be a clinical sample and can comprise at least one of blood, salvia, excreta, body tissue or tissue fluids.

Providing the at least one of the at least one measurement parameter to the at least one of the at least one measurement device can comprise transmitting a request for providing the at least one of the at least one measurement parameter to the at least one of the at least one measurement device to the interface module, and in response, the interface module providing the at least one of the at least measurement parameter to the at least one of the at least one measurement device.

The method further can further comprise controlling at least one of the at least one measurement device. The controlling the at least one of the at least one measurement device can comprise providing control data to the at least one of the at least one measurement device.

Providing the control data to the at least one of the at least one measurement device can comprise transmitting a request for providing the control data to the at least one of the at least one measurement device to the interface module, and in response, the interface module providing the control data to the at least one of the at least one measurement device.

The method can further comprise retrieving at least a portion of the analysis data from the data storage system. The at least a portion of the analysis data retrieved from the data storage system can comprise the at least a portion of the measurement data comprised by the analysis data.

The retrieving at least a portion of the analysis data from the data storage system can comprise transmitting a request for receiving the at least a portion of the analysis data to the interface module, in response, the interface module retrieving the requested data from the data storage system, and receiving a response comprising the requested data from the interface module.

The method can comprise triggering and/or initiating the step of analysing analysis data and/or pre-processing the measurement data prior to analysing the analysis data comprising at least a portion of the measurement data.

The storing data in the data storage system can comprise transmitting a request for storing the data to the interface module, and in response, the interface module prompting the data to be stored in the data storage system.

The data can comprise at least one of measurement data, analysis data, and/or the analysis result.

The triggering and/or initiating the step of analysing analysis data can be performed by the main module. The pre-processing of the measurement data prior to analysing the analysis data can be performed by the main module. The main module can utilize building blocks of the toolset for pre-processing the measurement data.

The transmitting of a request for storing the measurement data to the interface module can be performed by the measurement device. The interface module can be configured to define possible interactions between components of the system.

The method can comprise the interface module receiving a request from outside of the system and/or providing a response to a device, component and/or module outside of the system and/or upon request providing data to a device, component and/or module outside of the system.

At least a portion of the steps of the method or all of them can be performed automatically.

The method can further comprise identifying a faulty operation at least one of the at least one measurement device.

The method can comprise authenticating the origin of every request transmitted to the interface module. Additionally or alternatively, it can comprise providing a digital signature to the interface module with every request transmitted. The method can also comprise encrypting communication with the interface module.

The present invention is also defined by the following numbered embodiments.

Below, method embodiments will be discussed. These embodiments are abbreviated by the letter “M” followed by a number. Whenever reference is herein made to “method embodiments”, these embodiments are meant.
M1. A method for analysis in medical diagnostics, wherein the method comprises
   a. collecting measurement data,
   b. analysing analysis data corresponding to at least a portion of the measurement data and thereby generating an analysis result, and
   c. storing the measurement data and/or analysis data and/or analysis result in a data storage system,
   d. wherein the step of storing the measurement data and/or analysis data and/or analysis result in the data storage system comprises
      i. transmitting a request for storing the measurement data and/or analysis data and/or analysis result to an interface module, and
      ii. in response, the interface module prompting the measurement and/or analysis data and/or analysis result data to be stored in the data storage system.
M2. A method for analysis in medical diagnostics, wherein the method comprises
   a. providing a plurality of software-based toolsets in a data storage system, and
   b. in response to a query transfer one or more toolsets to a user.
M3. Combination of the methods according to the before method embodiments.
M4 The method according to the preceding method embodiment, wherein the method is a method for spectral analysis in medical diagnostics.
M5. The method according to any of the preceding method embodiments, wherein the medical diagnostics is metabolite diagnostics.
M6. The method according to any of the preceding system embodiments, wherein the medical diagnostics comprises diagnostics of human samples.
M7. The method according to any of the preceding method embodiments, wherein the medical diagnostics comprises diagnostics on animal samples.
M8. The method according to any of the preceding method embodiments, wherein the medical diagnostics comprise diagnostics of plant samples.
M9. The method according to any of the preceding method embodiments, wherein the method further comprises providing the sample.
M10. The method according to the preceding method embodiment, wherein the step of providing the sample comprises pre-processing the sample.
M11. The method according to any of the preceding method embodiments, wherein the sample is a clinical sample.
M12. The method according to the preceding method embodiment, wherein the sample comprises at least one of blood, salvia, excreta, body tissue or tissue fluids.
M13. The method according to any of the preceding method embodiments, wherein the measurement data comprise spectral data.
M14. The method according to the preceding method embodiment, wherein the spectral data comprises at least one nuclear magnetic resonance (NMR) spectrum.
M15. The method according to any of the preceding method embodiments, wherein collecting measurement data on the sample comprises performing a measurement on the sample utilizing at least one measurement device.
M16. The method according to the preceding method embodiment, wherein collecting measurement data on the sample comprises triggering a measurement of at least one of the at least one measurement device.
M17. The method according to any of the 2 preceding method embodiments, wherein the method further comprises initiating at least one of the at least one measurement device.
M18. The method according to any of the 3 preceding method embodiments, wherein the measurement on the sample is at least partially based on at least one measurement parameter of at least one of the at least one measurement device.
M19. The method according to the preceding method embodiment, wherein the method further comprises providing at least one of the at least one measurement parameter to the at least one of the at least one measurement device
M20. The method according to the preceding method embodiment, wherein providing the at least one of the at least one measurement parameter to the at least one of the at least one measurement device comprises
   a. transmitting a request for providing the at least one of the at least one measurement parameter to the at least one of the at least one measurement device to the interface module, and
   b. in response, the interface module providing the at least one of the at least measurement parameter to the at least one of the at least one measurement device
M21. The method according to any of the preceding method embodiments and with the features of M13, wherein the method further comprises controlling at least one of the at least one measurement device.
M22. The method according to the preceding method embodiment, wherein controlling the at least one of the at least one measurement device comprises providing control data to the at least one of the at least one measurement device.
M23. The method according to the preceding method embodiment, wherein providing the control data to the at least one of the at least one measurement device comprises
   a. transmitting a request for providing the control data to the at least one of the at least one measurement device to the interface module, and
   b. in response, the interface module providing the control data to the at least one of the at least one measurement device.
M24. The method according to any of the preceding method embodiments and with the features of M13, wherein the at least one measurement device is configured to transmit the measurement data to the interface module.
M25. The method according to any of the preceding method embodiments and with the features of M13, wherein the at least one measurement device is configured to communicate with the interface module.
M26. The method according to any of the preceding method embodiments, wherein the method further comprises retrieving at least a portion of the analysis data from the data storage system.
M27. The method according to the preceding method embodiments, wherein the at least a portion of the analysis data retrieved from the data storage system comprises the at least a portion of the measurement data comprised by the analysis data.
M28. The method according to any of the 2 preceding method embodiments, wherein retrieving at least a portion of the analysis data from the data storage system comprises
   a. transmitting a request for receiving the at least a portion of the analysis data to the interface module,
   b. in response, the interface module retrieving the requested data from the data storage system, and
   c. receiving a response comprising the requested data from the interface module.
M29. The method according to any of the preceding method embodiments, wherein the analysis data comprises subject data comprising information regarding the subject to which the sample is attributed and/or on which measurement data is collected.
M30. The method according to any of the preceding method embodiments and with the features of M16, wherein the analysis data comprises at least a subset of the at least one measurement parameter.
M31. The method according to any of the preceding method embodiments, wherein the measurement data comprises information indicative of the presence and/or abundance of metabolites in the sample.
M32. The method according to the preceding method embodiment, wherein analysing the analysis data comprises assessing the presence and/or abundance of certain metabolites and/or metabolite constellations in the sample.
M33. The method according to any of the preceding method embodiments, wherein the analysis result comprises a score indicative of a pre-defined condition.
M34. The method according to the preceding method embodiment and with the features of M30, wherein the score is based on the assessment of the presence and/or abundance of certain metabolites and/or metabolite constellations in the sample.
M35. The method according to any of the preceding method embodiments, wherein the method comprises triggering and/or initiating the step of analysing analysis data.
M36. The method according to any of the preceding method embodiments, wherein the method comprises pre-processing the measurement data prior to analysing the analysis data comprising at least a portion of the measurement data.
M37. The method according to any of the preceding method embodiments, wherein storing data in the data storage system comprises
   a. transmitting a request for storing the data to the interface module, and
   b. in response, the interface module prompting the data to be stored in the data storage system.
M38. The method according to the preceding method embodiment, wherein the data comprises at least one of measurement data, analysis data, and the analysis result.
M39. The method according to any of the preceding method embodiments, wherein the interface module comprises at least one endpoint communication endpoint.
M40. The method according to the preceding method embodiments, wherein at least one of the at least one communication endpoint is configured for providing access to the data storage system.
M41. The method according to any of the preceding method embodiments, wherein the interface module is configured to receive a request out of a pre-defined set of requests.
M42. The method according to any of the preceding method embodiments, wherein the interface module is configured to provide a response out of a pre-defined set of responses.
M43. system further comprises at least one analysis module.
M44. The method according to the preceding method embodiment, wherein analysing analysis data is performed by the analysis module.
M45. The method according to any of the preceding method embodiments, wherein the interface module is at least one application programming interface (API).
M46. The method according to any of the preceding method embodiments, wherein the method comprises providing a toolset, wherein the toolset comprises building blocks for processing and/or analysing the measurement and/or analysis data.
M47. The method according to the preceding method embodiment, wherein the method comprises utilizing building blocks of the toolset for analysing the analysis data.
M48. The method according to any of the 2 preceding method embodiments and with the features of M34, wherein the method comprises utilizing building blocks of the toolset to pre-process the measurement data.
M49. The method according to any of the 3 preceding method embodiments wherein a plurality of building blocks are configured for being combined to process and/or analyse the measurement and/or conduct the analyses together and/or any combination.
M50. The method according to any of the 3 preceding method embodiments, wherein the toolset is a software development kit (SDK).
M51. The method according to any of the preceding method embodiments, wherein the method utilizes a system for analysis in medical diagnostics, wherein the system comprises
   a. the interface module, and
   b. the data storage system.
M52. The method according to the preceding method embodiment and with the features of M13, wherein the system further comprises the at least one measurement device.
M53. The method according to any of the 2 preceding method embodiments and with the features of M24, wherein retrieving at least a portion of the analysis data from the data storage system is performed by the analysis module.
M54. The method according to any of the 3 preceding method embodiments and with the features of M26, wherein transmitting the request for receiving the at least a portion of the analysis data to the interface module is performed by the analysis module.
M55. The method according to any of the 3 preceding method embodiments and with the features of M26, wherein receiving the response comprising the requested data from the interface module is performed by the analysis module.
M56. The method according to any of the 4 preceding method embodiments and with the features of M42, wherein the method comprises the analysis module utilizing building blocks of the toolset for analysing the analysis data.
M57. The method according to any of the preceding method embodiments and with the features of M46, wherein the system further comprises a main module.
M58. The method according to the preceding method embodiment, wherein transmitting the request for storing the measurement data to an interface module is performed by the main module.
M59. The method according to any of the 2 preceding method embodiments and with the features of M14, wherein triggering the measurement of the at least one measurement device is performed by the main module.
M60. The method according to any of the 3 preceding method embodiments and with the features of M15, wherein initiating the at least one measurement device is performed by the main module.
M61. The method according to any of the 4 preceding method embodiments and with the features of M17, wherein providing the at least one of the at least one measurement parameter to the at least one of the at least one measurement device is performed by the main module.
M62. The method according to any of the 5 preceding method embodiments and with the features of M19, wherein controlling the at least one of the at least one measurement device is performed by the main module.
M63. The method according to any of the 6 preceding method embodiments and with the features of M20, wherein providing the control data to the at least one of the at least one measurement device is performed by the main module.
M64. The method according to any of the 7 preceding method embodiments and with the features of M21, wherein transmitting a request for providing the control data to the at least one of the at least one measurement device to the interface module is performed by the main module.
M65. The method according to any of the 8 preceding method embodiments and with the features of M33, wherein triggering and/or initiating the step of analysing analysis data is performed by the main module.
M66. The method according to any of the 9 preceding method embodiments and with the features of M34, wherein pre-processing of the measurement data prior to analysing the analysis data is performed by the main module.
M67. The method according to the preceding method embodiment and with the features of M42, wherein the method comprises the main module utilizing building blocks of the toolset for pre-processing the measurement data.
M68. The method according to any of the preceding method embodiments and with the features of M13, wherein transmitting a request for storing the measurement data to the interface module is performed by the measurement device.
M69. The method according to any of the preceding method embodiments and with the features of M46, wherein the interface module is configured to define possible interactions between components of the system.
M70. The method according to any of the preceding method embodiments and with the features of M46, wherein the method comprises the interface module receiving a request from outside of the system.
M71. The method according to any of the preceding method embodiments and with the features of M46, wherein the method comprises the interface module providing a response to a device, component and/or module outside of the system.
M72. The method according to any of the preceding method embodiments and with the features of M46, wherein the method comprises the interface module upon request providing data to a device, component and/or module outside of the system.
M73. The method according to any of the preceding method embodiments, wherein the method further comprises providing a user interface.
M74. The method according to the preceding method embodiment and with the features of M46, wherein the system comprises the user interface.
M75. The method according to any of the 2 preceding method embodiments, wherein the user interface comprises a visual interface.
M76. The method according to the preceding method embodiment, wherein the method comprises providing visual data via the visual interface.
M77. The method according to the preceding method embodiment, wherein the visual data comprises information on at least a portion of the measurement data.
M78. The method according to any of the preceding method embodiments, wherein the method further comprises receiving a user input.
M79. The method according to the preceding method embodiment and with the features of M70, wherein the user input is received by the user interface.
M80. The method according to any of the 2 preceding method embodiments and with the features of M54, wherein the user input is provided to the main module.
M81. The method according to any of the preceding method embodiments, wherein at least a portion of the steps of the method are performed automatically.
M82. The method according to the preceding method embodiment, wherein all steps of the method are performed automatically.
M83. The method according to any of the 2 preceding method embodiments, wherein automatically performed steps are at least partially performed by means of at least one processing unit.
M84. The method according to any of the preceding method embodiments and with the features of M13, wherein the method further comprises identifying a faulty operation at least one of the at least one measurement device.
M85. The method according to any of the preceding method embodiment, wherein the interface module is configured for secure communication.
M86. The method according to any of the preceding method embodiments, wherein the method comprises providing an authentication token to the interface module for any communication via the interface module.
M87. The method according to any of the preceding method embodiments, wherein the communication includes any type of data send to the interface module, particularly any request.
M88. The method according to any of the preceding method embodiments, wherein the method comprises authenticating the origin of every request transmitted to the interface module.
M89. The method according to any of the preceding method embodiments, wherein the method comprises providing a digital signature to the interface module with every request transmitted.
M90. The method according to any of the preceding method embodiments, wherein the method comprises encrypting communication with the interface module.
M91. The method according to any of the preceding method embodiments and with the features of M13 and M54, wherein the at least one measurement device is configured to transmit the measurement data to the main module.
M92. The method according to any of the preceding method embodiments, wherein the method further comprises providing an interface for connecting to a laboratory information management system (LIMS).
M93. The method according to any of the preceding method embodiments, further comprising the step of storing a modified toolset by a first user for downloading the modified first user toolset configured for a defined kind of measurement data.
M94. The method according to the preceding method embodiment wherein the modified first user toolset comprises at least one of:
   a. a first user originated toolset developed by the first user;
   b. a combination of toolsets and/or a modification of a toolset; and/or
   c. any one of measurement data, analysis data and analysis results established by the first user.
M95. The method according to the preceding method embodiment wherein the modified first user toolset is made available for other third-party users.
M96. The method according to the preceding method embodiment wherein the modified first user toolset is stored in a third-party toolset database.
M97. The method according to any of the 2 preceding method embodiments wherein a plurality of modified first user toolsets is catalogized in a third-party toolset database.
M98. The method according to any of the 5 preceding method embodiments further comprising an invoicing step on behalf of the first user to the third-party users when the third-party user downloads the first user toolset.
M99. The method according to the preceding method embodiment wherein the invoicing step on behalf of the first user is internally splitting up the invoice to the benefit of the first user and an organizing entity.
M100. The method according to the preceding method embodiment comprising an automatic payment by a third-party and transferring a pre-defined part of the payment to the first user and the rest of the payment to an organizing party.

Below, system embodiments will be discussed. These embodiments are abbreviated by the letter “S” followed by a number. Whenever reference is herein made to “system embodiments”, these embodiments are meant.
S1. A system for analysis in medical diagnostics, wherein the system comprises
   a main module,
   at least one measurement device configured to provide measurement data on a sample and/or subject,
   at least one analysis module configured to analyse analysis data and provide an analysis result,
   a data storage system configured to store and provide data, and
   an interface module.
S2. A system for analysis in medical diagnostics comprises a plurality of software-based toolsets in a data storage system wherein the system is configured to transfer one or more toolset(s) to a user in response to a query transfer.
S3. The system according to the two preceding embodiments.
S4. The system according to any of the preceding system embodiments wherein the data storage system is configured to store the measurement data and/or analysis data and/or analysis result.
S5. The system according to the preceding system embodiment, wherein the system is configured for spectral analysis in medical diagnostics.
S6. The system according to any of the preceding system embodiments, wherein the medical diagnostics is metabolite diagnostics.
S7. The system according to any of the preceding system embodiments, wherein the medical diagnostics comprises diagnostics of human samples.
S8. The system according to any of the preceding system embodiments, wherein the medical diagnostics comprises diagnostics of animal samples.
S9. The system according to any of the preceding system embodiments, wherein the medical diagnostics comprises diagnostics of plant samples.
S10. The system according to any of the preceding system embodiments, wherein the at least one measurement device comprises an imaging device.
S11. The system according to the preceding system embodiment, wherein the imaging device it at least one of a computer tomography scanner and a magnetic resonance imaging scanner.
S12. The system according to any of the preceding system embodiments, wherein the at least one measurement device comprises a spectrometer.
S13. The system according to the preceding system embodiment, wherein the at least one measurement device comprises a mass spectrometer.
S14. The system according to any of the 2 preceding system embodiments, wherein the at least one measurement device comprises a nuclear magnetic resonance (NMR) spectrometer.
S15. The system according to the preceding system embodiment, wherein the measurement data comprise NMR data.
S16. The system according to the preceding system embodiment, wherein the NMR data comprise at least one NMR spectrum.
S17. The system according to any of the preceding system embodiments, wherein the at least one measurement device is configured to communicate with the interface module.
S18. The system according to any of the preceding system embodiments, wherein the at least one measurement device is configured to exchange data with the interface module or more specifically to send data to and receive data from the interface module, in particular, a device, module and/or component that is said to be configured to communicate with the interface module is configured to transmit requests to and receive responses from the interface module.
S19. The system according to any of the preceding system embodiments, wherein the at least one measurement device is configured to transmit measurement data to the interface module.
S20. The system according to any of the preceding system embodiments, wherein the at least one measurement device is configured to directly communicate with the main module.
S21. The system according to the preceding system embodiments, wherein the at least one measurement device is configured to transmit measurement data to the main module.
S22. The system according to any of the preceding system embodiments, wherein the main module is configured to communicate with the interface module.
S23. The system according to any of the preceding system embodiments, wherein the main module is configured to directly communicate with the at least one measurement device.
S24. The system according to any of the preceding system embodiments, wherein the main module is configured to directly communicate with the at least one analysis module.
S25. The system according to any of the preceding system embodiments, wherein the at least one analysis module is configured to communicate with the interface module.
S26. The system according to any of the preceding system embodiments, wherein the at least one analysis module is configured to directly communicate with the main module.
S27. The system according to any of the preceding system embodiments, wherein the interface module is configured to transmit and receive data.
S28. The system according to any of the preceding system embodiments, wherein the interface module is configured to communicate with the at least one measurement device.
S29. The system according to any of the preceding system embodiments, wherein the interface module is configured to communicate with the main module.
S30. The system according to any of the preceding system embodiments, wherein the interface module is configured to communicate with the at least one analysis module.
S31. The system according to the preceding system embodiment, wherein the interface module is configured to communicate with the data storage system.
S32. The system according to any of the preceding system embodiments, wherein the interface module is configured to communicate with external devices and modules.
S33. The system according to any of the preceding system embodiments, wherein the interface module comprises at least one communication endpoint.
S34. The system according to the preceding system embodiments, wherein at least one of the at least one communication endpoint is configured for providing access to the data storage system.
S35. The system according to any of the preceding system embodiments, wherein the system comprises a plurality of interface modules.
S36. The system according to any of the preceding system embodiment, wherein the each of the plurality of interface modules comprises different communication endpoints.
S37. The system according to any of the preceding system embodiments, wherein the interface module is at least one application programming interface (API).
S38. The system according to any of the preceding system embodiments, wherein the data storage system comprises at least one database.
S39. The system according to any of the preceding system embodiments, wherein the data storage system comprises at least one data warehouse.
S40. The system according to any of the preceding system embodiments, wherein the data storage system comprises at least one remote server.
S41. The system according to any of the preceding system embodiments, wherein the data storage system is a remote, web-based data storage system.
S42. The system according to any of the preceding system embodiments, wherein the analysis data comprises measurement data.
S43. The system according to any of the preceding system embodiments, wherein the analysis data comprises subject data comprising information regarding the subject to which the sample is attributed.
S44. The system according to any of the preceding system embodiments, wherein a measurement performed by the measurement device to provide measurement data at least partially based on at least one measurement parameter.
S45. The system according to the preceding system embodiments, wherein the analysis data comprises at least a subset of the at least one measurement parameter.
S46. The system according to any of the preceding system embodiments, wherein the measurement data comprises information indicative for the presence and/or abundance of metabolites in the measured sample.
S47. The system according to the preceding system embodiment and with the features of S38, wherein the analysis module is configured to assessing the presence or abundance of certain metabolites and/or metabolite constellations in the sample.
S48. The system according to any of the preceding system embodiments, wherein the analysis result comprises a score indicative of a pre-defined condition.
S49. The system according to the preceding system embodiment and with the features of S43, wherein the score is based on the assessment of the presence or abundance of certain metabolites and/or metabolite constellations in the sample.
S50. The system according to any of the preceding system embodiments, wherein the main module is configured to trigger and/or initiate an analysis performed by at least one of the at least one analysis module.
S51. The system according to any of the preceding system embodiments, wherein the main module is configured to provide analysis parameters to the analysis module directly and/or via the interface module.
S52. The system according to the any of the preceding system embodiments, wherein the main module is configured to provide the analysis data to the analysis module directly and/or via the interface module.
S53. The system according to any of the preceding system embodiments, wherein the main module comprises a driver of at least one of the at least one measurement device.
S54. The system according to any of the preceding system embodiments, wherein the main module is configured to control the at least one measurement device.
S55. The system according to the preceding system embodiment, wherein the main module is configured to control the at least one measurement device by providing control data to the measurement device directly and/or via the interface module.
S56. The system according to any of the 2 preceding system embodiments, wherein the main module is configured to trigger and/or initiate a measurement of at least one of the at least one measurement device.
S57. The system according to any of the 3 preceding system embodiments and with the features of S40, wherein the main module is configured to provide and/or control at least a part of the at least one measurement parameter for a measurement of at least one of the at least one measurement device.
S58. The system according to any of the preceding system embodiments, wherein the system comprises at least one processing unit.
S59. The system according to the preceding system embodiment, wherein the main module is configured to run on at least one of the at least one processing unit.
S60. The system according to any of the 2 preceding system embodiments, wherein each of the at least one analysis module is configured to run on at least one of the at least one processing unit, respectively.
S61. The system according to any of the 3 preceding system embodiments, wherein the interface module is configured to run on at least one of the at least one processing unit.
S62. The system according to any of the 4 preceding system embodiments, wherein the measurement device comprises at least one of the at least one processing unit.
S63. The system according to any of the 5 preceding system embodiments, wherein the data storage system comprises at least one of the at least one processing unit.
S64. The system according to any of the preceding system embodiments, wherein the system comprises a toolset, wherein the toolset comprises building blocks for processing and/or analysing the measurement and/or analysis data.
S65. The system according to the preceding embodiment, wherein the toolset is comprised by the data storage.
S66. The system according to any of the 2 preceding embodiments, wherein the toolset is a software development kit (SDK).
S67. The system according to any of the 3 preceding embodiments, wherein the building blocks comprise algorithms for processing and/or analysing the measurement and/or analysis data.
S68. The system according to any of the 4 preceding embodiments, wherein the building blocks comprise pre-compiled routines and/or reusable components of programming code.
S69. The system according to any of the 5 preceding embodiments, wherein the toolset comprises a documentation comprising information regarding the building blocks.
S70. The system according to any of the 6 preceding embodiments, wherein the toolset comprises an application programming interface.
S71. The system according to any of the 7 preceding embodiments, wherein the toolkit comprises the interface module.
S72. The system according to any of the preceding system embodiments, wherein the sample is a clinical sample.
S73. The system according to any of the preceding system embodiments, wherein the sample comprises at least one of blood, salvia, excreta, body tissue or tissue fluids.
S74. The system according to any of the preceding system embodiments, wherein the system further comprises a user interface.
S75. The system according to the preceding system embodiment, wherein the user interface is configured to receive user input.
S76. The system according to the preceding system embodiment, wherein the main module is configured to receive the user input from the user interface.
S77. The system according to any of the 3 preceding system embodiments, wherein the user interface is configured to provide data to a user.
S78. The system according to any of the 4 preceding system embodiments, wherein the user interface comprises a visual interface.
S79. The system according to the preceding system embodiment, wherein the user interface is configured to provide visual data via the visual interface.
S80. The system according to any of the preceding system embodiments, wherein the main module is configured to pre-process the measurement data.
S81. The system according to any of the preceding system embodiments, wherein the interface module is configured for secure communication.
S82. The system according to any of the preceding system embodiments, wherein the interface module is configured to require authentication for any communication with the interface module.
S83. The system according to any of the preceding system embodiments, wherein the interface module is configured to require an authentication token to be provided to the interface module when communicating with the interface module.
S84. The system according to any of the preceding system embodiments, wherein the interface module is configured to require a digital signature to be provided to the interface module when communicating with the interface module.
S85. The system according to any of the preceding system embodiments, wherein communication with the interface module is encrypted.
S86. The system according to any of the preceding system embodiments, wherein the system is configured to perform a method for analysis in medical diagnostics according to any of the preceding method embodiments.
S87. The system according to any of the preceding system embodiments, wherein the system further comprises an interface for connecting to a laboratory information management system (LIMS).
S88. The system according to any of the preceding system embodiments further comprising a storage configured for a storing a toolset by a user for downloading the toolset for a defined kind of measurement data and/or measurement
S89. The system according to the preceding system embodiment further comprising a third party's platform for storing their toolset and/or measurement data
S90. The system according to any of the preceding system embodiments, further comprising a third-party toolset database storing a modified toolset by a first user for downloading the modified first user toolset configured for a defined kind of measurement data.
S91. The system according to the preceding system embodiment wherein the modified first user toolset comprises at least one of:
   a. a first user originated toolset developed by the first user;
   b. a combination of toolsets and/or a modification of a toolset; and/or
   c. any one of measurement data, analysis data and analysis results established by the first user.
S92. The system according to the preceding system embodiment wherein the modified first user toolset stored in the third-party toolset database is made available for other third-party users.
S93. The system according to any of the 2 preceding system embodiments wherein a plurality of modified first user toolsets is catalogized in the third-party toolset database.
S94. The system according to any of the 5 preceding system embodiments further comprising an invoicing step on behalf of the first user to the third-party users when the third-party user downloads the first user toolset.
S95. The system according to the preceding system embodiment wherein the invoicing step on behalf of the first user is internally splitting up the invoice to the benefit of the first user and an organizing entity.
S96. The system according to the preceding system embodiment with a payment component automatically settling the invoice from the third party and transferring a pre-defined part of the payment to the first user and the rest of the payment to an organizing party.

Below, use embodiments will be discussed. These embodiments are abbreviated by the letter “U” followed by a number. Whenever reference is herein made to “use embodiments”, these embodiments are meant.
U1. Use of the system according to any of the preceding system embodiments for medical diagnostics.
U2. Use of the system according to any of the preceding system embodiments for carrying out the method according to any of the preceding method embodiments.
U3. Use of the system according to any of the preceding system embodiments for NMR-based diagnostics.
U4. Use of the system according to any of the preceding system embodiments for metabolite diagnostics.

Below, computer related product embodiments will be discussed. These embodiments are abbreviated by the letter “C” followed by a number. Whenever reference is herein made to “computer related product embodiments”, these embodiments are meant.
C1. A computer program product comprising instructions, which, when the program is executed by a user interface device, causes a user device to perform the method steps according to any method embodiment, which have to be executed on the user interface device, wherein the user interface device is according to any system embodiment that comprises a user device that is compatible to said method embodiment.
C2. A computer program product comprising instructions, which, when the program is executed by a combination of a server and user interface device, cause the server and the user interface device to perform the method steps according to any method embodiment, which have to be executed on the server and the user interface device, wherein the user device and the server is according to any system embodiment that comprises a sever and/or the user device that is compatible to said method embodiment.
C3. A computer program product comprising instructions, which, when the program is executed by a server, cause the server to perform the method steps according to any method embodiment, which have to be executed on the server, wherein the server is according to any system embodiment that comprises a server that is compatible to said method embodiment.
C5. A computer related product with a program that is configured for carrying out the method according to any of the preceding method embodiments.
C6. A computer related product with a program that is trained for carrying out the method according to any of the preceding method embodiments.

Embodiments of the present invention will now be described with reference to the accompanying drawings. These embodiments should only exemplify, but not limit, the present invention.

### BRIEF DESCRIPTION OF THE DRAWINGS

FIG. 1 depicts a diagnostic system according to the state of the art;

FIG. 2A depicts an exemplary embodiment of a diagnostics system according to the present invention;

FIG. 2B depicts a further exemplary embodiment of a diagnostics system according to the present invention;

FIG. 3 depicts an exemplary embodiment of a diagnostics system comprising a plurality of measurement device and analysis modules; and

FIG. 4 schematically depicts steps of a method according to the present invention carried out on a system according to the present invention.

### DETAILED DESCRIPTION OF EMBODIMENTS

It is noted that not all the drawings carry all the reference signs. Instead, in some of the drawings, some of the reference signs have been omitted for the sake of brevity and simplicity of the illustration. Embodiments of the present invention will now be described with reference to the accompanying drawings.

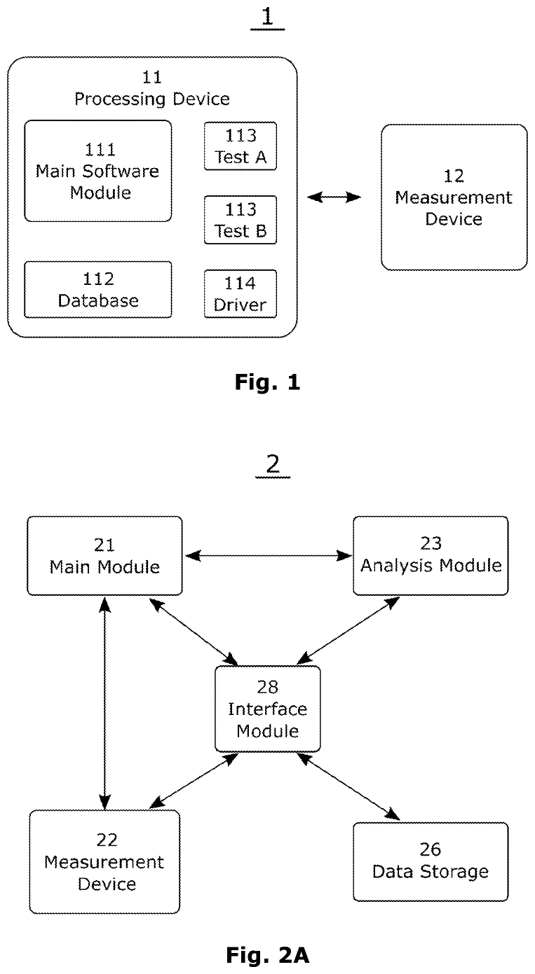

Reference will now be made to FIG. 1, depicting a diagnostic system 1 according to the state of the art. The diagnostic system 1 comprises a processing device 11 and a measurement device 12. Very generally the measurement device 12 may be configured to acquire measurement data, e.g. medical and/or clinical data which may provide basis for diagnosis, screening and/or monitoring of patients, as well as research purposes. For example, such a measurement device 12 may be a medical imaging device such as a computer tomography scanner (CT scanner), or a magnetic resonance imaging scanner (MRI scanner), or particularly a device for in-vitro diagnostics, e.g. a nuclear magnetic resonance (NMR) spectrometer.

The processing device 11 may comprise a main software module 111 which may be configured to control the measurement, as well as receiving, processing and/or storing the analysis data. That is, the processing device 11 may be configured to control the measurement device 12 and for example initiate a measurement, set and/or control parameters of a measurement and receive the respective measurement data. Thus, the processing device may for example comprise a respective driver 114 associated with the measurement device 12. The main software module 111 may for example be organized in a multitier architecture, e.g. a three-tier architecture which separates presentation, application and data layer, and provide a stand-alone desktop application.

Furthermore, the processing device 11 may comprise a local database 112 wherein for example measurement data may be stored, e.g. in a raw or processed format.

Furthermore, the processing device 11 may comprise one or more diagnostic test modules 113 (also referred to as test module 113, diagnostic test 113, or simply test 113), which are each a separate software module that may be accessed by the main software module 111 and/or by other modules. The individual test modules 113 are each designed to provide a designated result, e.g., a score, that may for example be indicative of a pre-defined disease. In particular, each test 113 may comprise a number of algorithms required to process and analyse the measurement data. Generally, the software-based tests 113 analyse the measurement data. For example, they may evaluate the presence or abundance of certain metabolites and/or metabolite constellations. Thus, the processing device 11 is also configured to analyse measurement data provided by the measurement device 12, e.g. by means of applying specific tests 113. Results of such tests may for example be utilized for diagnosis, prevention or monitoring of patients.

However, such a diagnostic system 1 may comprise certain disadvantages, firstly, the main software module 111 as well as the individual test modules 113 are each installed on a designated processing device 11, i.e. a hardware system, e.g. a PC, which runs a designated operating system, e.g. Windows. Further, any test module 113 that may be used to analyse measurement data must be locally installed on the processing device 11, e.g. as a plug-in software module. Thus, such a solution may lack flexibility and require all components to be matched to each other. In particular since according to the state of the art, the processing device 11 is typically a stand-alone device such as a computer, which is located close by, e.g. next to, the measurement device 12.

Furthermore, the system 1 only comprises a local database 112, which restricts access to the measurement data. It can also comprise a remote database (not shown) or a combination of both. Similarly, any measurement device 12 may typically require a designated processing system 11 and in particular a designated database 112 such that a test 113 may for example not readily have access to measurement data of different measurement devices 12. That is, the data are stored in a local database 112 on the same processing device 11 as the software 111, 113 and are also only available locally on the same processing device 11. This may cause problems when trying to access data from multiple, separate processing devices 11 since the databases 112 are locally stored and completely independent of each other and therefore not readily accessible from a central device, e.g. for comparing multiple data sets of different measurement devices 12.

Furthermore, each test module 113 comprises all algorithms required to process and analyse the data for that specific test 113. However, since each test 113 is provided as a stand-alone software module, there may be significant redundancies between different tests 113, e.g. test A and test B may partially rely on the same algorithms, particularly for initial processing of the measurement data. Thus, both tests 113 may at least partially comprise the same algorithms, which may lead to unnecessary redundancies. In addition, since there is no access to the individual algorithms a user may not rely on already developed and tested algorithms for developing a new test 113.

Therefore, a diagnostic system 1 as known in the state of the art may overall be inflexible and for example disadvantageously lack possibilities for combining measurement data of a plurality of measurement devices 12, for controlling a plurality of measurement devices 12, for remote storage and/or access to measurement data, and/or for general access to algorithms utilized by the tests 113 and/or the main software module 111.

With reference to FIG. 2A an exemplary diagnostic system 2 according to the present invention is discussed, which overcomes or at least alleviates shortcomings and disadvantages of the prior art. The diagnostic system 2 may comprise a main module 21, at least one measurement device 22, at least one analysis module 23, a data storage system 26 (also simply referred to as data storage 26) and an interface module 28.

Again, the at least one measurement device 22 may for example be an imaging device, e.g. a medical imaging device for providing measurement data on a subject, such as a CT scanner or an MRI scanner, or preferably a device for in-vitro diagnostics, which may provide measurement data on a sample, such as an NMR spectrometer or a mass spectrometer. In other words, the at least one measurement device 22 may generally be configured to provide measurement data on a sample and/or subject. The measurement data provided by the measurement device 22 may comprise NMR data, e.g. NMR data comprising an NMR spectrum. The subject may be a patient, e.g. a human or an animal, or for example an entire plant. The sample may generally be a human sample, an animal sample and/or a plant sample. Preferably, the sample may be a clinical sample, which may for example comprise at least one of blood, salvia, excreta, body tissue or tissue fluids of the respective subject.

In some embodiments, the measurement device 22 may comprise at least one processing unit, which may for example be configured to run the measurement device 22 according to measurement parameters and/or control data for example provided by external components, e.g. through communication with other components of the system, such as the interface module 28 and/or the main module 21. In general, the measurement device 22 may be configured to provide measurement data at least partially based on at least one measurement parameter. It will be understood that each measurement device 22 may also be configured to function without external control/control data. For example, while it may be possible for the main module 21 to control a measurement device 22, the measurement device 22 may generally also be configured to function without external control, e.g. it may only require triggering/initiating a measurement and only optionally be controlled externally.

The at least one measurement device 22 may be configured to directly communicate with the main module 21, e.g., to receive control data such as specific instructions for measurements and/or transmit measurement data. That is, the measurement device 22 may be configured to receive data from and transmit data to the main module 21. Additionally or alternatively, the measurement device 22 may be configured to communicate with the interface module 28. In particular, the measurement device 22 may be configured to transmit measurement data to the interface module 28 and/or receive data, e.g. control data, from the interface module 28. Such control data may preferably be provided by the main module 21. Thus, the measurement device 22 may generally be configured to communicate with the main module 21, i.e. via the interface module 28 and/or directly (i.e. without the interface module 28 being involved in the communication).

The main module 21 may be configured to control the measurement device 22. For example, the main module 21 may be configured to initiate and/or trigger a measurement of the measurement device 22. That is, the main module 21 may be configured to initiate and/or trigger, i.e. start, a measurement of the measurement device 22 on a respective sample and/or subject, wherein initiating the measurement may for example depend on the sample/subject to be measured. The main module 21 may be configured to provide and/or control at least a part of at least one measurement parameter of the measurement device 22 for a respective measurement. More generally, the main module 21 may be configured to provide control data to the measurement device 22, wherein the control data may for example comprise instructions for a measurement, measurement parameters, information on the sample, etc. In other words, the main module 21 may be configured to control the measurement device 22 by providing respective control data. In some embodiments the main module may comprise and/or have access to a driver of the at least one measurement device 22.

Thus, the main module 21 may generally be configured to communicate with the measurement device 22, again directly and/or via the interface module 28. That is, the main module 21 may for example be configured to directly transmit data to and receive data from the measurement device 22, e.g. control data and/or measurement data. Additionally or alternatively, the main module 21 may be configured to communicate with the interface module 28 and for example transmit and receive data via the interface module 28.

Additionally or alternatively, multiple measurement devices can be used. Another option is that one or more control center(s) manages all devices in the farms. However, the control center be could implemented in such a way that a driver is run on every device, so that the control center could be a browser application that could accessed from everywhere and simply pulls all required information from the driver instances running at the various devices of the farm.

The main module 21 may further be configured to store data, e.g. measurement data, analysis data and/or subject data, in the data storage system 26 via the interface module 28. Subject data may generally relate to the subject whereon a measurement is performed or to whom the measured sample may be associated.

Furthermore, the main module 21 may also be configured to communicate with the analysis module 23, e.g. directly and/or via the interface module 28. The main module 21 may for example be configured to provide analysis parameters and/or analysis data to the analysis module 23. Analysis data may be data to be analysed, which may for example comprise measurement data, subject data and/or measurement parameters. The main module may also be configured to initiate and/or trigger an analysis of respective analysis data, which may be provided by the main module 21 and/or alternatively retrieved from the data storage system 26. Furthermore, the main module 21 may for example (pre-) process measurement data prior to the analysis module 23 analysing the data.

Thus, the main module 21 may be configured to receive and/or (pre-) process measurement data provided by the measurement device 22 either via the interface module 28 or, in some embodiments, directly from the measurement device 22. The main module 21 may thus be configured to communicate with the interface module 28, e.g. to transmit control data, and/or to transmit and/or receive measurement data and/or (pre-) processed data. In some embodiments, the main module 21 may be configured to directly communicate with the measurement device 22, e.g. to allow for fast and secure direct control of the measurement device 22, without relying on the interface module 28. In some embodiments, the main module 21 may be configured to directly communicate with the at least one analysis module 23, e.g. it may provide data to the analysis module, initiate an analysis and/or set or provide analysis parameters to the analysis module 23.

The system 2 may generally also comprise a user interface 24 (not shown) and the main module 21 may be further configured to receive user input from the user interface 24, which may for example comprise a visual interface and/or be configured to receive user input data.

Generally, the main module 21 may be configured to run on at least one processing unit. That is, the main module 21 may be a software module which may be executed on at least one processing unit.

In other words, considering the main software module 111 known from the state of the art, which may comprise a three-tier architecture which separates presentation, application and data layer, the present system 2 may comprise a main module 21, which may for example provide the functionality the presentation layer and the application layer of the main software module 111, while the functionality of the data layer may be provided through the interface module 28.

The at least one analysis module 23 may generally comprise a functionality similar to the diagnostic tests modules 113 known from the prior art. That is, an analysis module 23 may generally be configured to analyse analysis data and to provide a designated analysis result, e.g. a score indicative of a pre-defined disease. For example, the at least one analysis module 23 may evaluate the presence or abundance of certain metabolites and/or metabolite constellations in a sample and respective analysis results may for example be utilized for diagnosis, prevention or monitoring of patients. Thus, the analysis module 23 may comprise a number of algorithms and/or have access to corresponding algorithms required for providing a respective analysis result. The analysis data may generally comprise at least some measurement data and for example additionally measurement parameters and/or subject data, comprising information regarding the subject to which the sample is attributed and/or on which measurement data is collected. The analysis module 23 may be configured to (pre-) process measurement data during analysing the analysis data comprising at least a portion of said measurement data.

Furthermore, the analysis module 23 may be configured to communicate with the interface module 28, i.e. to exchange data with the interface module 28. This may for example allow the analysis module 23 to gain access to measurement data to be analysed and/or transmit analysis results for inspection and/or storage. For example, the analysis module 23 may be configured to receive data, e.g. measurement data upon sending a corresponding request to the interface module 28. The analysis module 23 may thus be configured to retrieve data, e.g. measurement data, from the data storage system 26 via the interface module 28.

The analysis module can comprise an SDK that can comprise a collection of algorithms that can be used in various settings. Separate from that there can be an API 28 configured for remotely accessing our data. It can also require some sort of main module that addresses the before mentioned modules in order to do or trigger certain steps.

Furthermore, the analysis module 23 may be configured to exchange data with the main module 21, e.g. transmit data to and/or receive data from the main module 21. In particular, the main module 21 may for example trigger an analysis performed by at least one of the at least one analysis module 23 and/or receive an analysis result from the analysis module either via the interface module 28 or via direct communication. In some embodiments the main module 21 may also provide data to the analysis module 23, e.g. measurement data, measurement parameters, and/or subject data providing additional information about the subject which provided the sample that was measured in the measurement device 22. Again, such data may be provided via the interface module 28 or via a direct communication between the analysis module and the main module 21.

The analysis module 23 may be configured to run on at least one processing unit. That is, the analysis module 23 may be a software module that may provide the above discussed functionality when executed on at least one processing unit. In some embodiments, the analysis module 23 may for example be run on the same processing unit as the main module 21.

The data storage system 26 may generally be configured to store and retrieve data. Very generally, the data storage system 26 may for example comprise at least one database and/or data warehouse and/or data store. In particular, the data storage system 26 may be configured to communicate with the interface module 28 to receive data from other system components, e.g. from the main module 21, the measurement device 22 and/or the analysis module 23 and/or to provide (e.g. transmit) data stored in the data storage system 26 to other system components. Thus, system components that are configured to communicate with the interface module 28, i.e. system components that can send data to and/or receive data from the interface module 28, may transmit data to and/or retrieve data from the data storage system 26 via the interface module 28. The data may for example be measurement data which may be raw (i.e. directly from the measurement device 22) and/or (pre-) processed, measurement parameters, e.g. settings of the measurement device 22, subject data and/or analysis results.

Since the communication with other system components is limited to the interface module 28, the data storage system 26 and/or components thereof may for example be in a remote location, e.g. the data storage system 26 may be realised utilising a cloud-based architecture. This may advantageously allow for a greater flexibility in design of the data storage system 26, for better accessibility and/or increased independence of other system components.

The interface module 28 may generally be configured to communicate with other system components and, in some embodiments, with devices and components outside of the diagnostic system 2. That is, the interface module 28 may be configured to receive and transmit data from and to other system components and in some embodiments, devices and components outside of the diagnostic system 2. In other words, the interface module 28 may permit communication and interaction between different (system) components/devices. In particular, the interface module 28 may allow other devices and components to access the data storage system 26 for storing and retrieving data. Advantageously, this may allow for a standardized communication with the data storage system according to a set of pre-defined rules and data formats.

In particular, the interface module 28 may comprise at least one, and preferably a plurality of endpoints, configured for communicating via the interface module 28. In other words, an endpoint may constitute the end of a communication channel of the interface module 28. Thus, other system components may for example send requests to an endpoint of the interface module 28 and correspondingly receive a respective response.

The API can standardize the way how data is accessed. Therefore, it is both the access point to the data storage, but also an option to realize other components that need data (e.g., live monitoring apps, statistical analytics apps, etc.). In addition, the API can take care of authentication and authorization, as some applications as e.g. live monitoring apps will never have a need to store data, but only to retrieve data.

For example, the measurement device 22 may send a request for storing measurement data in the data storage 26, which may include the measurement data to be stored. The interface module 28 may then transmit the measurement data to the data storage 26 alongside with instructions to store the respective measurement data. Upon successful storing of the measurement data, the data storage 26 may confirm the success to the interface module 28, which may in turn send a response to the measurement device 22 indicating successful storage of the measurement data in the data storage 26. Similarly, one of the at least one analysis module 23 may request measurement data that is to be analysed via the interface module 28, which may in turn retrieve the respective measurement data form the data storage system 26 and transmit it to the analysis module 23 in response to the respective request.

Very generally, storing data in the data storage system 26, e.g. measurement data, an analysis result, or subject data, may thus comprise transmitting a respective request to the interface module 28, e.g. to a designated endpoint of the interface module 28, and in response, the interface module 28 prompting the data to be stored in the data storage system 26, e.g. by transmitting the data to be stored alongside a corresponding request to store the data to the data storage system 26. Optionally the data storage system 26 and/or the interface module 28 may return a response indicating successful storage and/or an error that may have occurred in the process.

Similarly retrieving data from the data storage system 26, e.g. measurement data, an analysis result or subject data, may comprise transmitting a respective request for retrieving the data to the interface module 28, in response, the interface module 28 retrieving the requested data from the data storage system 26, e.g. through designated requests/commands, and the interface module 28 providing a response comprising the requested data retrieved from the data storage system 26.

Thus, whenever it is said, that a system component, device and/or module, is configured to communicate with the interface module 28, this relates to said component, device and/or module being configured to transmit requests to and receive responses from the interface module 28. More generally this may also be referred to as transmit data to and receive data from the interface module 28, since it will be understood that any request and/or response is represented by certain data.

In some embodiments, there may be different endpoints for providing different functionalities, e.g. an endpoint designated to storing data, e.g. measurement data, an endpoint designated to controlling/accessing measurement devices 22 etc. It will be understood, that the diagnostic system 2 may comprise a plurality of interface modules 28, wherein each interface module 28 provides for communication/interaction related to different resources, e.g. one interface module 28 for controlling and accessing the measurement device, one interface module 28 for accessing the data storage and one interface module 28 for performing an analysis utilizing one of the at least one analysis modules 23. In other words, it will be understood that a single interface module 28 may be replaced by a plurality of interface modules 28, wherein each of the plurality of interface modules 28 may provide at least a subset of the functionality of the single interface module 28 discussed with respect to the figures. Thus, the combination of the plurality of interface modules 28 may provide the same functionality as the interface module 28 described herein with respect to the figures.

In some embodiments, the interface module 28 may be an application programming interface (API).

Generally, the interface module 28 may enable a greater flexibility and versatility of the diagnostic system 2. With reference to FIG. 2B it may for example allow for including a plurality of measurement devices 22, 22A, 22B, 22C into the diagnostic system 2, which may all be controlled by a single main module 21. Said control may for example be provided via the interface module 28. However, the main module 21 and the analysis module 23 may additionally or alternatively also be configured to directly communicate (not shown), such that a control of the measurement devices 22, 22A, 22B, 22C may not require the interface module 28. For example, control data may be sent directly to the measurement devices 22, 22A, 22B, 22C and/or measurement data may be directly transmitted from the measurement devices 22, 22A, 22B, 22C to the main module 21. Generally, the measurement devices 22, 22A, 22B, 22C may for example be in remote locations, e.g. a measurement device farm comprising a plurality of measurement devices, or each at a separate location wherein one or more measurement devices 22, 22A, 22B, 22C may also be in close vicinity to the main module 21, e.g. to a processing unit on which the main module is running, e.g. executed.

Similarly, also a plurality of analysis modules 23, 23A, 23B, 23C may be comprised by the system 2, which may readily access data stored in the data storage 26, e.g. data of a plurality of measurement devices 22, 22A, 22B, 22C. Generally, the analysis modules 23, 23A, 23B, 23C may be accessible via the interface module 28, i.e. communication such as triggering an analysis, providing analysis data (or parts thereof) to the analysis modules 23, 23A, 23B, 23C, retrieving and/or storing an analysis result, may be performed through the interface module 28. Again, the analysis modules 23, 23A, 23B, 23C and the main module 21 may further be configured to directly communicate with each other (not shown). Such that the main module 21 may for example directly provide analysis data to an analysis module 23, 23A, 23B, 23C and/or initiate or trigger a respective analysis.

Generally, it will be understood that the same considerations apply as for the embodiment described with respect to FIG. 2A.

Particularly due to the communication link via the interface module 28, the individual system components may be independent of each other. The interface module 28 may for example be configured to also be accessed via a remote connection, e.g. a LAN connection or the internet. Thus, the incorporation of an interface module 28 into the diagnostic system 2 may advantageously allow to scale the diagnostic system 2 and ensure that communication between all components is established/maintained.

In particular, the interface module 28 may further advantageously provide platform-independent communication in that it may provide a number of allowed interactions, e.g, methods and/or functions, that may be independent of the platform utilized by the component, e.g. module or device communicating/interacting with the interface module 28. This may advantageously allow to modify and/or exchange any component of the diagnostic system 2, while still enabling communication therewith as long as communication with the interface module 28 is maintained, which may be based on accessing an endpoint and utilizing any of the pre-defined interactions. In other words, the interface module 28 may ensure that communication follows a known protocol and for example provides data in a pre-defined, known structure. Thus, if for example the data storage 26 is modified, e.g. to run on a different operating system or change internal methods for storing and retrieving data, the data may still be accessible to any other system component as long as correct communication of the data storage 26 with the interface module 28 is maintained. In other words, the measurement device 22 does not need any information on how data is stored in the data storage system 26 and/or for example on which operating system the data storage system 26 runs, it merely requires knowledge on how to communicate with the interface module 28. Thus, if anything is changed in the data storage system 26 the measurement device 22 may not require any information about these changes, let alone require any changes. In other words, the interface module 28 may provide an abstract way, e.g. protocol, for communicating with different system components and particularly for accessing the data storage 26. Thus, the interface module 28 may enable independent development, exchange and/or addition of system components since communication is standardized, disclosed and/or published through the interface module 28 and its pre-defined allowed interactions and endpoints.

In other words, the interface module 28 may for example provide a possibility for system components or even external devices to access the data storage system 26, e.g. store and/or retrieve data from the data storage system 26, without requiring any details on the operations necessary to actually access the particular data storage system 26.

Some of the advantages that can be provided by an interface module 28, e.g. an API, are increased security, authentication for access to the data storage system 26, different levels of authorization for accessing to the data storage system 26, e.g. limiting access to only required/allowed portions of the data stored in the data storage system depending on the component/device accessing the data storage system 26, scalability of the diagnostic system and abstraction and/or simplification of communication within the system.

However, it will be understood, that some system components may also be configured to directly communicate (not shown in FIG. 2B). For example, as outlined above, the main module 21 may be configured to directly communicate with at least one of the at least one measurement device 22, 22A, 22B, 22C as for example indicated by arrow in FIG. 2A. That is, some components/devices of the diagnostic system 2 may be configured to directly communicate and/or the interface module 28 may in some such embodiments not be configured to also provide equivalent communication functionalities. In other words, in some embodiments the interface module 28 may for example not be configured to provide communication between the main module 21 and at least some of the measurement devices 22, 22A, 22B, 22C, which may instead be configured to directly communicate.

The interface module 28 may not be limited to local communication but advantageously also enable remote and/or distant communication, for example via a network, such as a local area network (LAN) and/or the internet. This may advantageously allow for the main module 21, the at least one analysis module 23, the data storage system 26 and the at least one measurement device 22 not to be in close proximity. For example, a plurality of measurement devices 22, 22A, 22B, 22C may be comprised by the diagnostic system 2, wherein the measurement devices 22, 22A, 22B, 22C are located separately from each other and for example even in remote locations such as different laboratories.

Furthermore, since the interface module 28 may also allow for external access to system components, it may for example allow for remote monitoring of a measurement device 22. For example, it may allow to track the progress of a current measurement and/or notifications of errors in case any appear throughout a measurement.

Very generally, modules may be software based and run by a processing unit. That is modules may be constituted by a software/programming code that, when executed by a processing unit provides the functionality of the respective module. Generally, modules may be run by different processing units or on the same processing unit.

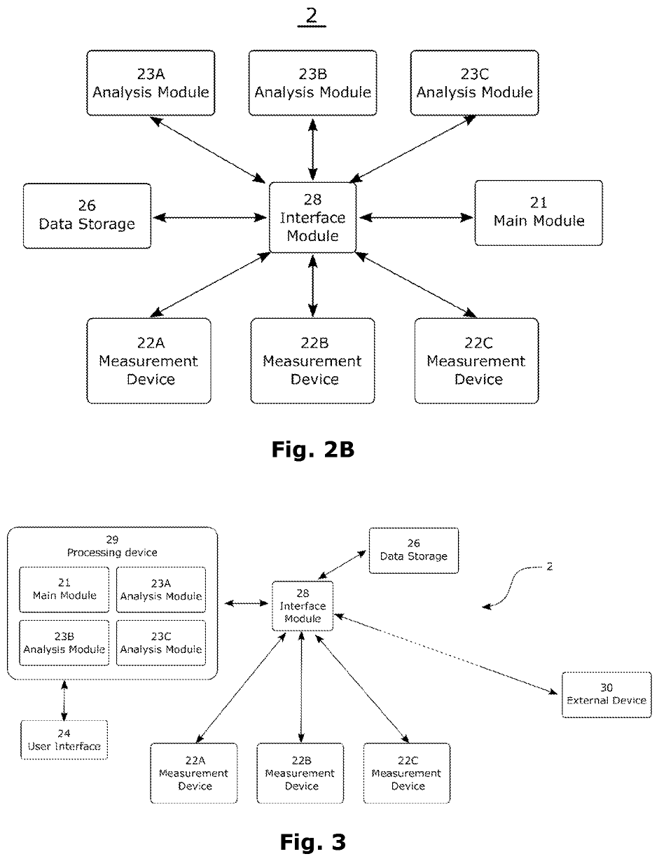

With respect to FIG. 3 an exemplary embodiment of the present invention is shown, wherein the main module 21 and a plurality of analysis modules 23, 23A, 23B, 23C may be run by the same processing unit and/or on the same processing device 29, while for example the data storage system 26 may be in a remote location and only accessible via the interface module 28, i.e. the data storage system 26 may be independent of the processing device 29. However, the system 2 may for example also only comprise one analysis module 23 or a plurality of analysis modules 23, 23A, 23B, 23C, wherein not all analysis modules 23 are run on the same processing unit and/or processing device. Referring to FIG. 3 again, in such a diagnostic system 2 the main module 21 and analysis modules 23, 23A, 23C, 23B may for example be configured to directly communicate instead of communicating via the interface module 28. However, alternatively or additionally, they may also be configured to communicate via the interface module 28. That is, due to the modularity and the interlink of components through the interface module 28 (and/or direct communication channels between some of the system components) the diagnostic system 2 may become highly flexible.

The diagnostic system may for example further comprise a user interface 24, which may for example be comprised by or configured to communicate with the processing device 29. Generally, the user interface 24 may be configured to communicate with the main module 21, e.g. provide user input to the main module 21, and or provide data, e.g. visual data, to the user via the user interface. A user interface 24 may thus for example comprise a visual interface, e.g. a screen, and/or be configured to receive input from a user, e.g. through a touch screen, a keyboard or a computer mouse.

The interface module 28 may be configured to communicate with an external device 30, that does not form part of the diagnostic system 2. Such an external device may for example be another processing device, e.g. a handheld and/or portable device, a desktop or a server. This may for example allow for live monitoring of an ongoing measurement of a measurement device 22, 22A, 22B, 22C, (pre-) viewing of an analysis result of an analysis module 23, 23A, 23B, 23C and/or access to data in the data storage system 26.

A respective method according to the present invention comprises the steps of collecting measurement data 50 on a sample and/or subject, storing the measurement data 50 in a data storage system 26, and analysing analysis data 70 comprising at least a portion of the measurement data 50 and thereby generating an analysis result 80, wherein the step of storing the measurement data 50 in the data storage system 26 comprises transmitting a request for storing the measurement data 50 to an interface module and, in response, the interface module prompting the measurement data 50 to be stored in the data storage system 26.

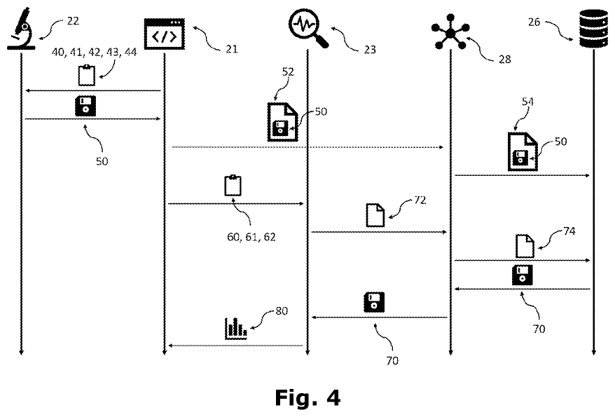

With respect to FIG. 4 an exemplary embodiment of a method utilizing a system 2 as described above is discussed. The step of collecting measurement data 50 on a sample and/or subject may comprise the main module 21 transmitting data 40 to the measurement device 21. For example, the method may comprise the main module 21 triggering a measurement of the measurement device 22 (or, more generally, of at least one of the at least one measurement device 22) and/or initiating the measurement device 22. For example, the main module 21 may directly communicate with the measurement device 22 and transmit a respective initiation request 41 for initiating the measurement device and/or a trigger request 42 for triggering of a measurement. Additionally or alternatively, control data 43 for controlling the measurement device 22 and/or at least one measurement parameter 44 may be transmitted from the main module 21 to the measurement device 22.

The measurement device 22 may then transmit resulting measurement data 50 to the main module 21. That is for example after collecting the measurement data 50 on a sample and/or subject or also already during the collection of measurement data 50, the measurement data 50 may be transmitted to the main module 21. In alternative embodiments, the measurement device 22 may transmit the measurement data 50 to another system component, e.g. to the interface module 28 or the analysis module 23.

As part of the step of storing the measurement data 50 in a data storage system 26, a request 52 for storing the measurement data 50 may be submitted to the interface module 28. In the depicted embodiment said store request 52 may be transmitted by the main module 21. The store request 52 may typically comprise the data to be stored, e.g. in this case the measurement data 50. In response to receiving the store request 52, the interface module 28 may prompt the measurement data 50 to be stored in the data storage system 26. For example, the interface module 28 may transmit the measurement data 50 to the data storage system 26 in a format that is accepted by and/or optimized for the data storage system 26, typically along with a corresponding storage request 54 to store the measurement data 50. In other words, the interface module 28 may forward the store request 52 to the data storage system 26 as a storage request 54. However, the interface module 28 may be configured to ensure that the request 54 is in the format that is expected by the data storage system 26. For example, the interface module 28 may translate the request into a set of commands understood by the data storage system 26 and/or arrange the measurement data 50 in a format that is accepted by and/or optimized for the data storage system 26. The data storage system 26 and/or the interface module may respectively provide a response to the request indicating successful storage and/or an error that occurred (not shown).

After successful storage of the measurement data 50, the main module 21 may for example trigger and/or initiate the step of analysing analysis data 70, wherein the main module may transmit data 60 to the analysis module 23, e.g. a request 61 for starting an analysis and/or analysis parameters 62 which may comprise at least a portion of the measurement data 50.

The analysis data 70 to be analysed may comprise at least a portion of the measurement data 50 and for example subject data and/or at least a subset of the at least one measurement parameter. The analysis module 23 may retrieve at least a portion of the analysis data 70 from the data storage system 26 (some of the analysis data 70 may for example also be provided by the main module 21, e.g. subject data and/or measurement parameter). Thus, the analysis module 23 may transmit a data request 72 to the interface module 28, for receiving at least a portion of the analysis data 70, e.g. all of the analysis data 70, from the data storage system 26.

In response to the data request 72, the interface module 28 may retrieve the requested data from the data storage system 26. For example, the interface module may send a data retrieval request 74 to the data storage system 26, specifying which data should be provided by the data storage system 26. Again, the interface module 28 may particularly ensure that the retrieval request is in the correct format for the data storage system. This may allow the analysis module 23 to retrieve data from the data storage system 26 without knowing the exact format for a request to the data storage system 26. That is, the analysis module may only require knowledge on how to communicate with the interface module 28, e.g. which requests are accepted by the interface module. This may also advantageously limit the access to the data storage system 26 and allow for a platform independent communication between the data storage system 26 and other system components.

The data storage system 26 may then provide the requested analysis data 70 to the interface module 28, which may in turn provide the analysis data 70 to the analysis module 23, e.g. in a response that is received by the analysis module 23 and comprises the requested analysis data 70 (or a requested portion of the analysis data 70).

After receiving the requested analysis data 70 the analysis module 23 may analyse the analysis data 70 and thereby generate an analysis result 80. The measurement data 50 may for example comprise information that may be indicative of the presence and/or abundance of metabolites in the measured sample. In such a case, analysing the analysis data may comprise accessing the presence and/or abundance of certain metabolites and/or metabolite constellations in the sample. Such information may for example be indicative of certain pre-defined diseases. Generally, the analysis result 80 may comprise a score indicative of a pre-defined condition, which may for example be based on the assessment of the presence and/or abundance of metabolites and/or metabolite constellations in the sample.

The analysis module 23 may for example transmit the analysis result 80 to the main module 21. Additionally or alternatively, the analysis result 80 may be stored in the data storage system 26 by transmitting a corresponding request comprising the analysis result 80 to the interface module 28. For example, the analysis module 23 or the main module 21 may transmit the respective request to the interface module 28, i.e. prompt/initiate storage of the analysis result in the data storage system 26.

It will be understood that the above is merely an example on how the method according to the preceding invention can be carried out by a corresponding diagnostic system 2. IN particular some of the steps may be performed by other components and/or additional steps may be included in the method.

For example, in some embodiments the main module 21 may (pre-) process the measurement data 50 before storing it in the data storage system 26.

Since measurement and/or analysis data relating to a subject, e.g. a patient, is typically highly confidential, the interface module may further configured to ensure a high level of security for the communication between system components and particularly for access to the data storage system 26. That is, the interface module 23 may generally be configured for secure communication. For example, the interface module 23 may require the provision of an authentication token for any communication via the interface module, which may allow to authenticate the origin of each request/communication transmitted to the interface module 23. Thus, the interface module 23 may restrict access to certain components/data depending on the authentication token. In some embodiments, the interface module 23 may for example require a digital signature for any request transmitted to the interface module. For example, the request may be transmitted as a digitally signed request, e.g. a hash of the request may be encrypted using a private key and submitted along the request such that the interface module 23 may verify the digital signature using a corresponding public key.

Additionally or alternatively the communication with the interface module may be encrypted, e.g. an encrypted communication channel may be established between the interface module, e.g. an endpoint thereof, and a component, e.g. device or module, communicating with the interface module.

Furthermore, the diagnostic system 2 according to the present invention may comprise a toolset which may comprise building blocks for processing and/or analysing the measurement and/or analysis data. That is, the toolset may generally provide building blocks that may provide certain functionalities required or desirable for processing and/or analysing data. Such building blocks may for example comprise algorithms for processing and/or analysing measurement data and/or analysis data. In particular, the building clocks of the toolset may comprise pre-compiled routines and/or reusable components of programming code. Thus, the toolset may advantageously allow to reduce redundancies in the system by providing the building blocks to multiple system components, such that for example, multiple analysis modules 23, 23A, 23B, 23C may at least partially utilizes the same building blocks of the toolset. This may further advantageously allow a user to use building blocks such as established routines and pre-compiled algorithms to build a new analysis module 23, e.g. to test for a desired signature in the measurement data such as a specific metabolite constellation. Therefore, the toolset may not only allow to reduce redundancies in the system but further also to provide the building blocks to a user for developing new analysis modules 23 while advantageously relying on established and tested building blocks, e.g. for general (pre-) processing of the measurement data and/or basic analysis steps. The toolkit may for example in some embodiments also comprise an application programming interface and/or the interface module. Furthermore, the toolkit may provide a documentation comprising information regarding the comprised building blocks and their respective functionality. However, the toolset may advantageously allow to not reveal the algorithms themselves, but only allow usage thereof, e.g. by providing the algorithms in a compiled format. This may advantageously allow to keep the exact functionality secret and/or only require the user to know input and output formats.

The toolset may for example simply be a library of functions that can be used by a user. In some embodiments the toolset may be a framework or a software development kit (SDK). Again, the toolset may thus advantageously allow a user to develop their own analysis modules 23 and/or reduce redundancies in the system by allowing modules to access building blocks of the toolset.

Whenever a relative term, such as “about”, “substantially” or “approximately” is used in this specification, such a term should also be construed to also include the exact term. That is, e.g., “substantially straight” should be construed to also include “(exactly) straight”.

Whenever steps were recited in the above or also in the appended claims, it should be noted that the order in which the steps are recited in this text may be accidental. That is, unless otherwise specified or unless clear to the skilled person, the order in which steps are recited may be accidental. That is, when the present document states, e.g., that a method comprises steps (A) and (B), this does not necessarily mean that step (A) precedes step (B), but it is also possible that step (A) is performed (at least partly) simultaneously with step (B) or that step (B) precedes step (A). Furthermore, when a step (X) is said to precede another step (Z), this does not imply that there is no step between steps (X) and (Z). That is, step (X) preceding step (Z) encompasses the situation that step (X) is performed directly before step (Z), but also the situation that (X) is performed before one or more steps (Y1), . . . , followed by step (Z). Corresponding considerations apply when terms like “after” or “before” are used.

While in the above, a preferred embodiment has been described with reference to the accompanying drawings, the skilled person will understand that this embodiment was provided for illustrative purpose only and should by no means be construed to limit the scope of the present invention, which is defined by the claims.

1. A method for analysis in medical diagnostics, wherein the method comprises
a. collecting measurement data,
b. analysing analysis data corresponding to at least a portion of the measurement data and thereby generating an analysis result, and
c. storing the measurement data and/or analysis data and/or analysis result in a data storage system,
d, wherein the step of storing the measurement data and/or analysis data and/or analysis result in the data storage system comprises
i. transmitting a request for storing the measurement data and/or analysis data and/or analysis result to an interface module, and
ii. in response, the interface module prompting the measurement and/or analysis data and/or analysis result data to be stored in the data storage system.

2. The method according to claim 1, wherein the method comprises
e. providing a plurality of software-based toolsets in a data storage system, and
f. in response to a query transfer one or more toolsets to a user.

3. (canceled)

4. The method according to claim 2, wherein the method is a method for spectral analysis in medical diagnostics.

5. The method according to claim 1, wherein the medical diagnostics is metabolite diagnostics.

6. The method according to claim 1, wherein the measurement data comprise spectral data.

7. The method according to claim 6, wherein the spectral data comprises at least one nuclear magnetic resonance (NMR) spectrum.

8. The method according to claim 1, wherein collecting measurement data on a sample comprises performing a measurement on the sample utilizing at least one measurement device.

9. The method according to claim 8, wherein collecting measurement data on the sample comprises triggering a measurement of at least one of the at least one measurement device.

10. The method according to claim 1, wherein the measurement on the sample is at least partially based on at least one measurement parameter of at least one of the at least one measurement device.

11. The method according to claim 10, wherein the method further comprises providing at least one of the at least one measurement parameter to the at least one of the at least one measurement device.

12. The method according to claim 11, wherein providing the at least one of the at least one measurement parameter to the at least one of the at least one measurement device comprises
g. transmitting a request for providing the at least one of the at least one measurement parameter to the at least one of the at least one measurement device to the interface module, and
h. in response, the interface module providing the at least one of the at least measurement parameter to the at least one of the at least one measurement device.

13. The method according to claim 1, wherein the at least one measurement device is configured to transmit the measurement data to the interface module.

14. The method according to claim 1, wherein the at least one measurement device is configured to communicate with the interface module.

15. The method according to claim 1, wherein the method further comprises retrieving at least a portion of the analysis data from the data storage system.

16. A system for analysis in medical diagnostics, wherein the system comprises a main module,
at least one measurement device configured to provide measurement data on a sample and/or subject,
at least one analysis module configured to analyse analysis data and provide an analysis result,
a data storage system configured to store and provide data, and an interface module.

17. The system according to claim 16, wherein the system comprises a plurality of software-based toolsets in a data storage system wherein the system is configured to transfer one or more toolset(s) to a user in response to a query transfer.

18. (canceled)

19. The system according to claim 16 wherein the data storage system is configured to store the measurement data and/or analysis data and/or analysis result.

20. The system according to claim 19, wherein the system is configured for spectral analysis in medical diagnostics.

21. The system according to claim 16, wherein the medical diagnostics is metabolite diagnostics.

22. The system according to claim 16, wherein the at least one measurement device comprises an imaging device.

23. The system according to claim 22, wherein the imaging device is at least one of a computer tomography scanner and/or a magnetic resonance imaging scanner.

24. The system according to claim 16, wherein the at least one measurement device comprises a spectrometer.

25. The system according to claim 24, wherein the at least one measurement device comprises a mass spectrometer.

26. The system according to claim 24, wherein the at least one measurement device comprises a nuclear magnetic resonance (NMR) spectrometer.

27. The system according to claim 26, wherein the measurement data comprise NMR data.

28. The system according to claim 27, wherein the NMR data comprise at least one NMR spectrum.
